(19) United States
(12) Patent Application Publication
(10) Pub. No.: US 2024/0119819 A1
(43) Pub. Date: Apr. 11, 2024

(54) NON-CONTACT BABY MONITORING USING ARTIFICIAL INTELLIGENCE

(71) Applicant: Covidien LP, Mansfield, MA (US)

(72) Inventors: Paul S. ADDISON, Edinburgh (GB); Dean MONTGOMERY, Edinburgh (GB)

(21) Appl. No.: 18/465,095

(22) Filed: Sep. 11, 2023

Related U.S. Application Data
(60) Provisional application No. 63/379,087, filed on Oct. 11, 2022.

(51) Int. Cl. G08B 21/02; A61B 5/00; G06T 7/50; G06T 7/70; G06V 10/25; G06V 20/52; G06V 40/16

(52) U.S. Cl. CPC G08B 21/0208; A61B 5/7405; A61B 5/742; A61B 5/746; G06T 7/50; G06T 7/70; G06V 10/25; G06V 20/52; G06V 40/166; G06T 2207/10016; G06T 2207/30201; G06T 2207/30232

(57) ABSTRACT

Use of non-contact monitoring systems to monitor a subject (e.g., baby, infant, child) in a sleeping or resting environment. The systems utilize artificial intelligence (AI) to identify potential hazards to the subject and alert a caregiver. The systems can be trained to recognize the location of an object as a potential hazard, recognize the location of an object as not a potential hazard, and/or to recognize the type of object as not a potential hazard. The caregiver may acknowledge the potential hazard is indeed a hazard or may clear the potential hazard and the alert, which the system will remember for subsequent like occurrences.

### CROSS-REFERENCE TO RELATED APPLICATIONS

The present application claims benefit of priority to U.S. Provisional Patent Application No. 63/379,087, entitled “NON-CONTACT BABY MONITORING USING ARTIFICIAL INTELLIGENCE” and filed on Oct. 11, 2022, which is specifically incorporated by reference herein for all that it discloses or teaches.

### BACKGROUND

Baby monitors are well known. Such monitors typically have either or both video cameras and audio microphones that are placed in close proximity to the child which transmit a signal to a remote monitor (video and/or audio speaker) to provide the caregiver with a visual and/or audio signal. From the signal, the caregiver can determine if the child is uncomfortable or in distress. Unfortunately, the caregiver is advised of discomfort or distress by the audio emanating from the child. Typical monitors fail to provide any indication, e.g., if the child stops breathing, which occurs with Sudden Infant Death Syndrome (SIDS), or if the child is in a precarious position.

Accordingly, there is a need for a better child monitoring system.

### SUMMARY

The present disclosure is directed to using non-contact monitoring systems to monitor a subject (e.g., baby, infant, child) in a sleeping or resting environment. The systems utilize artificial intelligence (AI) to identify potential hazards to the subject and alert a caregiver. The caregiver may acknowledge the potential hazard is indeed a hazard or may clear the potential hazard and the alert, which the system will remember for subsequent like occurrences.

One particular embodiment described herein is a method of monitoring a subject. The method includes detecting a location of the subject with a non-contact monitoring system utilizing depth measurements, detecting an object in a zone proximate the subject with the non-contact monitoring system utilizing depth measurements, determining, with the non-contact monitoring system, if the detected object satisfies one or more criteria designating the detected object as a potential hazard to the subject, and upon determining if the detected object satisfies one or more criteria designating the detected objection as a potential hazard to the subject, initiating an alarm.

Another particular embodiment described herein is another method of monitoring a subject with a non-contact monitoring system. The method includes detecting a location of the subject in a region of interest (ROI), detecting an object proximate the subject, determining, with the non-contact monitoring system, if the detected object satisfies one or more criteria designating the detected object as a potential hazard to the subject, wherein the non-contact monitoring system utilizes artificial intelligence (AI) to determine if the detected object satisfies one or more of the criteria, and upon determining the detected object satisfies one or more of the criteria and is therefore a potential hazard to the subject, initiating an alarm.

Other embodiments are also described and recited herein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

These and other aspects of the technology described herein will be apparent after consideration of the Detailed Description and Drawing herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWING

FIG. 1A is a simulated image of a monitored subject in an acceptable situation;

FIG. 1B is a simulated image of the monitored subject showing a potential hazard to the subject; and FIG. 1C is a simulated image of the monitored subject showing another potential hazard to the subject.

FIG. 2 is a schematic diagram of an example non-contact monitoring system.

FIG. 3 is a schematic diagram of another example non-contact monitoring system.

FIG. 4 is a block diagram of a non-contact monitoring system including a computing device, a server, and an image capture device according to various embodiments described herein.

FIG. 5 is a step-wise method for monitoring a subject.

### DETAILED DESCRIPTION

As described above, the present disclosure is directed to monitoring a subject (e.g., baby, infant, toddler) while resting or sleeping. Using the non-contact monitoring systems described herein mitigates the risk factors for suffocation, Sudden Infant Death Syndrome (SIDS), physical entanglement, and other threats by detecting the subject's position and the nearby presence of physical objects, such as the proximity of clutter (e.g., blanket, stuffed animal or toy, etc.) to the subject, particularly the subject's head and face. The non-contact monitoring systems can additionally determine the position of the subject (e.g., prone (lying on the front face down) or supine (lying on the back)) and the location of the subject (e.g., against the side of a crib, e.g., too close to crib padding). The systems can be used in a residential setting or in a medical or commercial setting, such as a hospital.

The non-contact monitoring systems use a video signal of the subject, identifying physiologically relevant areas within the video image (such as the subject's head, face, neck, arms, legs, or torso) and vision-based artificial intelligence (AI) methods to learn to identify potential hazards present in the relevant areas. A potential hazard can be one that is determined to satisfy one or more criteria that are provided to establish the likelihood of the detected object being a hazard or potential hazard to the subject. For example, the criteria used to determine if a detected object is a hazard or potential hazard can include, whether the object is closer than 12 inches to the subject, whether the object is closer than 6 inches to the subject, whether the object is covering some or all of a portion of certain portions of a subject's body (e.g., head or face), whether the object has a specific size and/or shape, etc. Using the video image, the systems extract a distance or depth signal from the relevant area, correlate the depth signals to the presence of or lack of an object, and use that indication to determine a potential threat to the subject (such as by assessing the object against the one or more criteria). In some embodiments, the systems correlate a change in depth signals over time to motion or the introduction of an object.

With the non-contact monitoring systems, signals representative of the topography and optionally movement of the subject are detected by a camera or camera system that views but does not contact the subject. The camera or camera system may utilize any or all of depth signals, color signals (e.g., RGB signals), and IR signals. With appropriate selection and filtering of the signals detected by the camera, the physiologic contribution by each of the detected signals can be isolated and measured.

Remote sensing of a subject with video-based monitoring systems, in general, often presents several challenges. One challenge is ambient light. In this context, “ambient light” means surrounding light not emitted by components of the camera or the monitoring system. In some embodiments of the non-contact monitoring system, the desired physiologic signal is generated or carried by a light source. Thus, because of this, the ambient light cannot be entirely filtered, removed, or avoided as noise. Changes in lighting within the room, including overhead lighting, sunlight, television screens, nightlights, variations in reflected light, and passing shadows from moving objects all contribute to the light signal that reaches the camera. Even subtle motions outside the field of view of the camera can reflect light onto the subject being monitored.

The present disclosure describes methods of non-contact monitoring of a subject to determine and alert of potential hazards due to the subject's position and/or undesired objects in close proximity to the subject (e.g., closer than 12 inches to the subject). The methods are particularly useful for alerting caregivers (e.g., parents) of a subject's (e.g., child's, e.g., infant's) position in bed or if there is a potential risk for suffocation or SIDS, e.g., if an object is too close to the subject's head or face, or if an object falls into the bed.

The non-contact monitoring systems used for the non-contact monitoring of the subject are developed to identify features of the subject and to identify objects whose location and/or position may pose a threat to the subject. The non-contact monitoring systems utilize AI to learn what objects and what positions/locations may pose a threat. Additionally, the non-contact monitoring systems can identify a new presence of an object. Upon determining a potential threat, the systems provide an alert to the caregiver.

The non-contact systems receive a video signal from the subject and the environment and from that extract a distance or depth signal from the relevant area to provide a topographical map from the depth signal; the systems may also determine any movement or motion from the depth signal. The systems can also receive a second signal, a light intensity signal reflected from the subject and environment, and from the reflected light intensity signal calculate a depth or distance and also a movement or motion. In some embodiments, the light intensity signal is a reflection of a pattern or feature (e.g., using visible color or infrared) projected onto the subject, such as by a projector.

The depth sensing feature of the system provides a measurement of the distance or depth between the detection system and the subject. One or two video cameras may be used to determine the depth, and change in depth, from the system to the subject. When two cameras, set at a fixed distance apart, are used, they offer stereo vision due to the slightly different perspectives of the scene from which distance information is extracted. When distinct features are present in the scene, the stereo image algorithm can find the locations of the same features in the two image streams. However, if an object is featureless (e.g., a smooth surface with a monochromatic color), then the depth camera system may have difficulty resolving the perspective differences. By including an image projector to project features (e.g., in the form of dots, pixels, etc., visual or IR) onto the scene, this projected feature can be monitored over time to produce an estimate of location and any change in location of an object.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

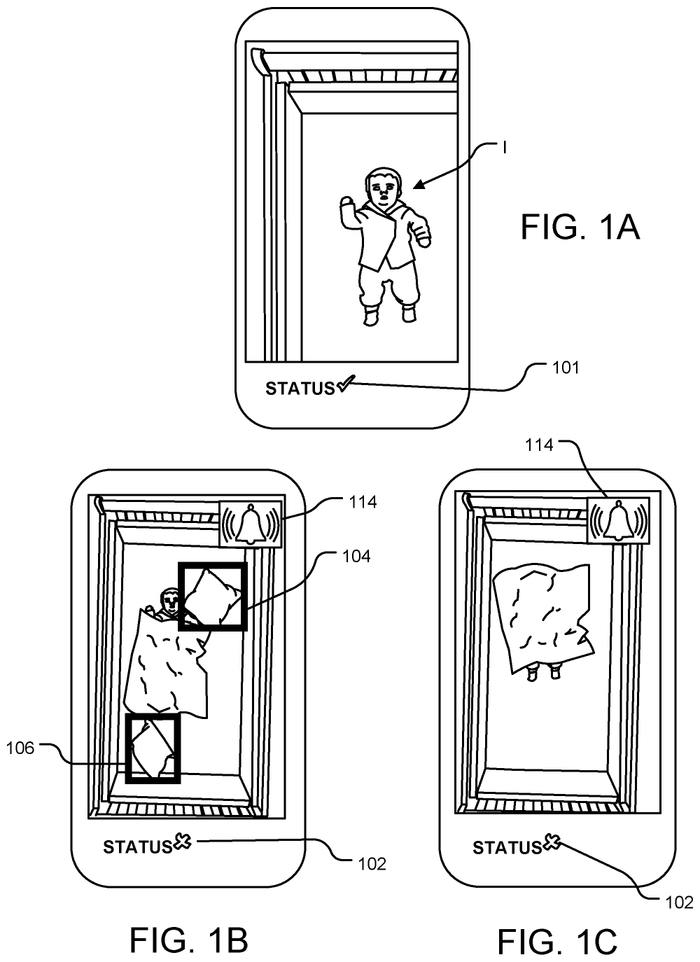

FIGS. 1A, 1B and 1C show example simulated images from non-contact monitoring systems of this disclosure, the images illustrating various situations detectable by the systems. The images are simulated images on a device such as a cell phone or tablet showing a subject I (particularly in these images, an infant) in a crib.

In FIG. 1A, the infant is seen centered in the crib. As the system has not found any potential hazards proximate the infant, a “good status” is indicated by the checkmark icon 101 in the image.

In FIG. 1B, the infant is seen covered in a blanket with a pillow proximate the child's head and another pillow proximate the child's feet. A “warning status” is indicated by the X icon 102 in the image. The pillow proximate the head is within a zone identified as potentially hazardous (e.g., within 12 inches of the subject, e.g., within 6 inches of the subject) and is delineated with a square 104, as the system has determined that this object is a potential hazard that warrants an alert (e.g., by virtue of the detected object satisfying one or more criteria used to designate a detected object as a potential hazard; in this case, the criteria being the object being within 12 or 6 inches of the subject). The delineating feature may be a warning color, e.g., red or orange. The pillow proximate the feet is also delineated with a rectangle 106, as the system has monitored the presence of the object within the zone identified as potentially hazardous but has determined that this pillow is not a potential hazard due to its location at the feet of the subject (e.g., by virtue of the detected object not satisfying one or more criteria used to designate a detected object as a potential hazard; in this case, the criteria being the object not being within 12 or 6 inches of the subject or not being within 12 or 6 inches of the subject's head or face). The delineating feature may be, e.g., green or blue. For both pillows, the system has been programmed, e.g., using vision-based artificial intelligence (AI) methods, to identify potential hazards present in the relevant areas, e.g., within a predetermined zone proximate at least to the head and face, based on, in some cases, satisfying one or more hazard criteria. Examples of items that could be potentially hazardous due to, e.g., their proximity to the subject include pillows, stuffed animals, plush toys, padding or bumpers on the crib sides.

The zone of proximity, for use as part of identifying whether an object is a potential hazard to the subject, may be present at the head/face/neck/shoulder region of the subject and may include the torso or even the entire subject. The zone may be, e.g., within 12 inches, within 6 inches, or within 3 inches of the subject, including touching the subject. The zone is programmed into the monitoring system and may be adjusted, e.g., manually, by the caregiver, for example, as the subject ages.

In FIG. 1C, the infant is seen covered with the blanket to the extent that the blanket covers the head. The system has determined that this object is a potential hazard due to, e.g., this condition satisfying one or more hazard criteria, and therefore warrants an alert. A warning status is indicated by the X icon 102 in the image. The system has been programmed to recognize that the subject's head and/or face are no longer identifiable, e.g., by using the color or IR image or through the topography determined using the depth camera.

In both situations, FIG. 1B and FIG. 1C, the system issues an alert to the caregiver. In this embodiment, an alert is indicated by the bell icon 114 in FIGS. 1B and 1C; the alert may be visual (e.g., the bell icon 114) and/or may be audible.

The system can be programmed to not alarm or alert for certain, approved, objects even if in close proximity to the subject's head/face, objects such as pacifiers, teething rings, and the subject's hands.

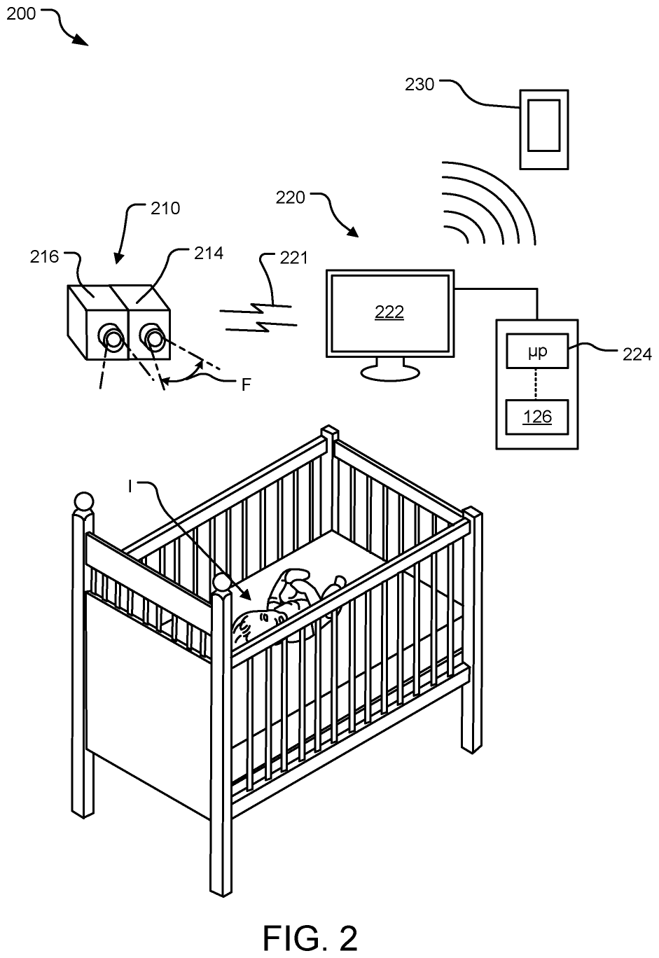

FIG. 2 shows a non-contact subject monitoring system 200 and a subject I, in this particular example an infant in a crib. It is noted that the systems and methods described herein are not limited to a crib, but may be used with a bassinette, an isolate, or any other place where the subject is left alone. The system 200 includes a non-contact detector system 210 placed remote from the subject I. In this embodiment, the detector system 210 includes a camera system 214, particularly, a camera that includes an infrared (IR) detection feature. The camera 214 may be a depth sensing camera, such as a Kinect camera from Microsoft Corp. (Redmond, Washington) or a Real Sense™ D415, D435 or D455 camera from Intel Corp. (Santa Clara, California). The camera system 214 is remote from the subject I, in that it is spaced apart from and does not physically contact the subject I. The camera system 214 may be positioned in close proximity to or on the crib. The camera system 214 includes a detector exposed to a field of view F that encompasses at least a portion of the subject I.

The camera system 214 includes a depth sensing camera that can detect a distance between the camera system 214 and objects in its field of view F. Such information can be used, as disclosed herein, to determine that a subject is within the field of view of the camera system 214 and determine a region of interest (ROI) to monitor on the subject. Once an ROI is identified, that ROI can be monitored over time, and the depth data can be used to locate the presence of an object (e.g., the subject I) and a change in depth of points can represent movements of the subject I or of objects in the ROI. The field of view F is selected to be at least the upper torso of the subject. However, as it is common for young children and infants to move within the confines of their crib, bed or other sleeping area, the entire area potentially occupied by the subject I (e.g., the crib) may be the field of view F. The ROI may be the entire field of view F or may be less than the entire field of view F.

The camera system 214 may operate at a set frame rate, which is the number of image frames taken per second (or other time period). Example frame rates include 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

The distance from the ROI on the subject I to the camera system 214 is measured by the system 200. Generally, the camera system 214 detects a distance between the camera system 214 and the surface within the ROI; the change in depth or distance of the ROI can represent movements of the subject or presence of an object in the ROI, e.g., a stuffed animal falling on the subject I.

In some embodiments, the system 200 determines a skeleton outline of the subject I to identify a point or points from which to extrapolate the ROI. For example, a skeleton may be used to find a center point of a chest, shoulder points, waist points, hands, head, and/or any other points on a body. These points can be used to determine the ROI. In other embodiments, instead of using a skeleton, other points are used to establish an ROI. For example, a face may be recognized, and a torso and waist area inferred in proportion and spatial relation to the face.

In another example, the subject I may wear a specially configured piece of clothing that identifies points on the body such as the torso or the arms. The system 200 may identify those points by identifying the indicating feature of the clothing. Such identifying features could be a visually encoded message (e.g., bar code, QR code, etc.), or a brightly colored shape that contrasts with the rest of the subject's clothing, etc. In some embodiments, a piece of clothing worn by the subject may have a grid or other identifiable pattern on it to aid in recognition of the subject and/or their movement. In some embodiments, the identifying feature may be stuck on the clothing using a fastening mechanism such as adhesive, a pin, etc., or stuck directly on the subject's skin, such as by adhesive. For example, a small sticker or other indicator may be placed on a subject's hands that can be easily identified from an image captured by a camera.

In some embodiments, the system 200 may receive a user input to identify a starting point for defining an ROI. For example, an image may be reproduced on an interface, allowing a user of the interface to select a point on the subject from which the ROI can be determined (such as a point on the head). Other methods for identifying a subject, points on the subject, and defining an ROI may also be used.

However, if the ROI is essentially featureless (e.g., a smooth surface with a monochromatic color, such as a blanket or sheet covering the subject I), then the camera system 214 may have difficulty resolving the perspective differences. To address this, the system 200 can include a projector 216 to project individual features (e.g., dots, crosses or Xs, lines, individual pixels, etc.) onto objects in the ROI; the features may be visible light, UV light, infrared (IR) light, etc. The projector may be part of the detector system 210 or the overall system 200.

The projector 216 generates a sequence of features over time on the ROI from which is monitored and measured the reflected light intensity. A measure of the amount, color, or brightness of light within all or a portion of the reflected feature over time is referred to as a light intensity signal. The camera system 214 detects the features from which this light intensity signal is determined. In an embodiment, each visible image projected by the projector 216 includes a two-dimensional array or grid of pixels, and each pixel may include three color components—for example, red, green, and blue (RGB). A measure of one or more color components of one or more pixels over time is referred to as a “pixel signal,” which is a type of light intensity signal. In another embodiment, when the projector 216 projects an IR feature, which is not visible to a human eye, the camera system 214 includes an infrared (IR) sensing feature. In another embodiment, the projector 216 projects a UV feature. In yet other embodiments, other modalities including millimeter-wave, hyper-spectral, etc., may be used.

The projector 216 may alternately or additionally project a featureless intensity pattern (e.g., a homogeneous, a gradient or any other pattern that does not necessarily have distinct features, or a pattern of random intensities). In some embodiments, the projector 216, or more than one projector, can project a combination of a feature-rich pattern and featureless patterns on to the ROI.

The light intensity of the image reflected by the subject surface is detected by the detector system 210.

The measurements (e.g., depth signal, RGB reflection, light intensity) are sent to a computing device 220 through a wired or wireless connection 221. The computing device 220 includes a display 222, a processor 224, and hardware memory 226 for storing software and computer instructions. Sequential image frames of the subject I are recorded by the video camera system 214 and sent to the computing device 220 for analysis by the processor 224. The display 222 may be remote from the computing device 220, such as a video screen positioned separately from the processor and memory. Other embodiments of the computing device 220 may have different, fewer, or additional components than shown in FIG. 2. In some embodiments, the computing device may be a server. In other embodiments, the computing device of FIG. 2 may be connected to a server. The captured images (e.g., still images or video) can be processed or analyzed at the computing device and/or at the server to create a topographical map or image to identify the subject I and any other objects with the ROI.

In some embodiments, the computing device 220 is operably connected (e.g., wirelessly, via WiFi connectivity, cellular signal, Bluetooth™ connectivity, etc.) to a remote device 230 such as a smart phone, tablet, or merely a screen. The remote device 230 can be remote from the computing device 220 and the subject I, for example, in an adjacent or nearby room. The computing device 220 may send a video feed to the remote device 230, showing e.g., the subject I and/or the field of view F. Additionally or alternately, the computing device 220 may send instructions to the remote device 230 to trigger an alarm, such as when the system 200 detects that an object is in a problematic position or location, causing a potential hazard to the subject I.

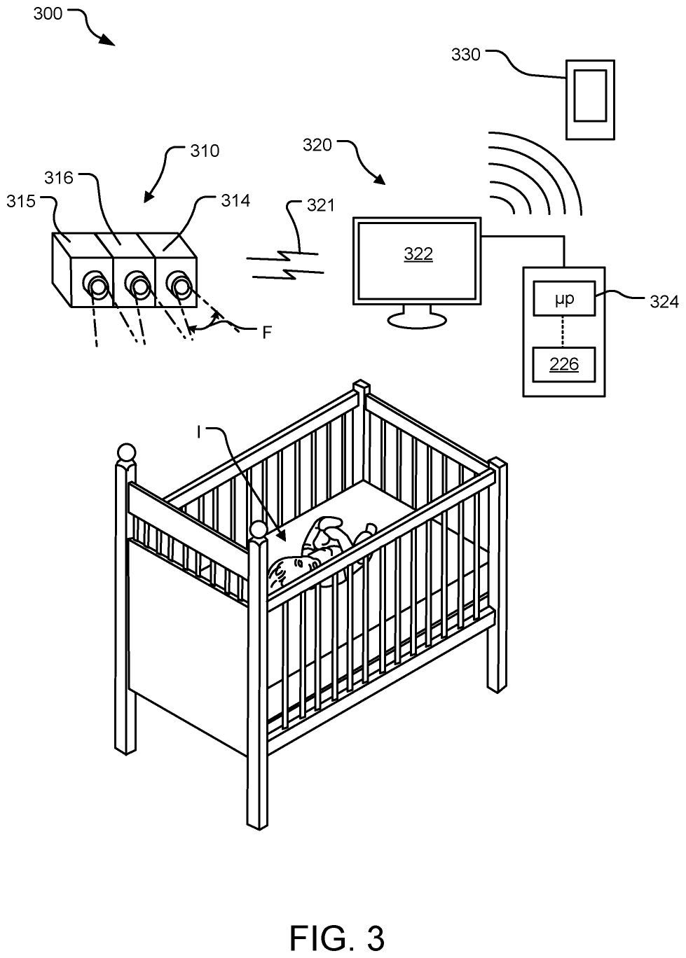

FIG. 3 shows another non-contact subject monitoring system 300 and a subject I, in this example, an infant in a crib. The system 300 includes a non-contact detector 310 placed remote from the subject I. In this embodiment, the detector 310 includes a first camera 314 and a second camera 315, at least one of which includes an infrared (IR) camera feature. The cameras 314, 315 are positioned so that their ROIs at least intersect, in some embodiments, completely overlap. The detector 310 also includes an IR projector 316, which projects individual features (e.g., dots, crosses or Xs, lines, or a featureless pattern, or a combination thereof etc.) onto the subject I in the ROI. The projector 316 can be separate from the detector 310 or integral with the detector 310, as shown in FIG. 3. In some embodiments, more than one projector 316 can be used. Both cameras 314, 315 are aimed to have features projected by the projector 316 to be in their ROI. The cameras 314, 315 and projector 316 are remote from the subject I, in that they are spaced apart from and do not contact the subject I. In this implementation, the projector 316 is physically positioned between the cameras 314, 315, whereas in other embodiments it may not be so.

The distance from the ROI to the cameras 314, 315 is measured by the system 300. Generally, the cameras 314, 315 detect a distance between the cameras 314, 315 and the projected features on a surface within the ROI. The light from the projector 316 hitting the surface is scattered/diffused in all directions; the diffusion pattern depends on the reflective and scattering properties of the surface. The cameras 314, 315 also detect the light intensity of the projected individual features in their ROIs. From the distance and the light intensity, the presence of the subject I and any objects are monitored, as well as any movement of the subject I or objects.

The detected images, diffusion measurements and/or reflection pattern are sent to a computing device 320 through a wired or wireless connection 321. The computing device 320 includes a display 322, a processor 324, and hardware memory 326 for storing software and computer instructions. The display 322 may be remote from the computing device 320, such as a video screen positioned separately from the processor and memory. In other embodiments, the computing device of FIG. 3 may be connected to a server. The captured images (e.g., still images or video) can be processed or analyzed at the computing device and/or at the server to create a topographical map or image to identify the subject I and any other objects with the ROI.

In some embodiments, the computing device 320 is operably connected (e.g., wirelessly, via WiFi connectivity, cellular signal, Bluetooth™ connectivity, etc.) to a remote device 330 such as a smart phone, tablet, or merely a screen. The remote device 330 can be remote from the computing device 320 and the subject I, for example, in an adjacent or nearby room. The computing device 320 may send a video feed to the remote device 330, showing, e.g., the subject I and/or the field of view F. Additionally or alternately, the computing device 320 may send instructions to the remote device 230 to trigger an alarm, such as when the system 300 detects that an object is in a problematic position or location, causing a potential hazard to the subject I.

For both systems 200, 300 and variants thereof, the computing device 220, 320 identifies whether any of the objects within the ROI are sufficiently proximate to the head and/or face of the subject I.

The computing device 220, 320 determines, from the image of the ROI (formed from the, e.g., depth signal, RGB reflection, light intensity measurements), the location and position of the head and/or face of the subject. The computing device 220, 320 then determines, from the image, if any objects are sufficiently proximate to the head and/or face of the subject to warrant an alarm. If a hazard is identified (see, e.g., FIG. 1B where two potential hazards are indicated), the computing device 220, 320 initiates an alarm on the remote device 230, 330.

The computing device 220, 320 can be trained with vision-based artificial intelligence (AI) methods to learn to identify objects in the image, including the face and/or head of the subject and other objects such as pillows, stuffed animals, etc. The computing device 220, 320 can also be trained with AI to determine if there are potential hazards in the relevant areas. The computing device 220, 320 can be trained using any standard AI model and standard methods, e.g., utilizing numerous data points to create a dataset of images. The system 200 300 can be configured to learn whether an object identified by the computing device 220, 320 is indeed a potential hazard.

For example, referring to FIG. 1B, the computing device 220, 320 has identified two potential hazards in the scene, a pillow proximate the head of the subject I identified in the square 104 and a pillow proximate the feet of the subject identified in the rectangle 106. Using repeated data entry, the computing device 220, 320 can be taught to identify the pillows as an object that can pose a potential hazard to the subject. The system 200, 300 can then alert a caregiver via an alarm (audible and/or visual) on the remote device 230, 330.

In some instances, an object may not be a hazard even if identified proximate the subject. Using that same data entry, the computing device 220, 320 can be taught that an object, although within the field of view and proximate the subject, is not a hazard when it is sufficiently removed from the subject's head/face, as is the pillow identified in the rectangle 106, which is proximate the subject's feet. Additionally or alternately, a user may manually override the computing device 220, 320 if the computing device 220, 320 determines that the object warrants an alert. An override may be a one-time override or the computing device 220, 320 can save the instructions and apply that override to subsequent similarly located objects.

The computing device 220, 320 can be trained to identify an object proximate the subject yet to accept the object as not hazardous. For example, the system can be trained to not trigger an alarm when a pacifier or teething ring is located proximate the head/face of the subject.

The computing device 220, 320 also determines, from the image of the ROI (formed from the, e.g., depth signal, RGB reflection, light intensity measurements), if an image of the head and/or face of the subject is not identified; see, e.g., FIG. 1C where the subject's head is covered with a blanket. If no head and/or face is identified, e.g., due to the lack of topographical features that represent a face or a portion of a head (e.g., an ear), the computing device 220, 230 initiates an alarm on the remote device 230, 330.

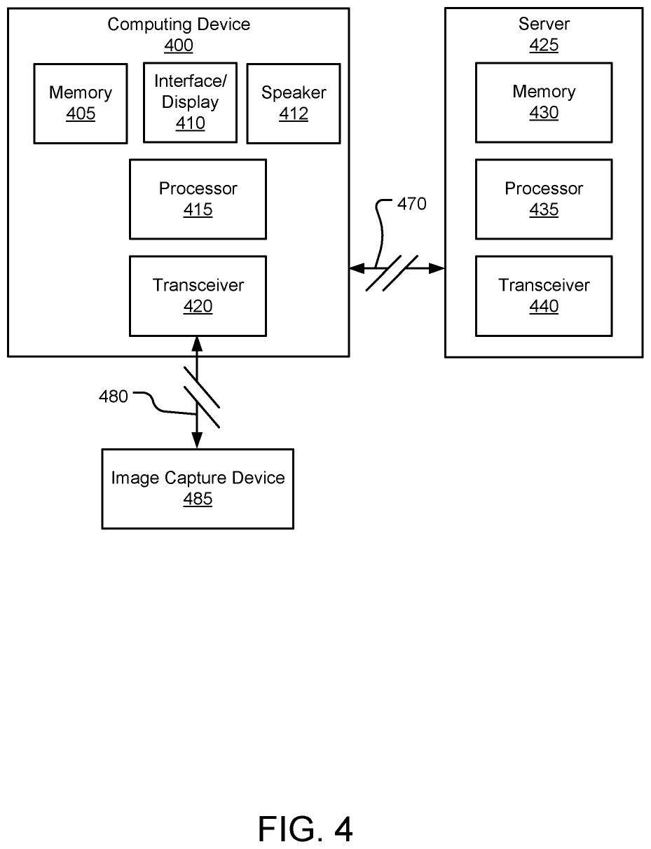

The computing device 220, 320 has an appropriate memory, processor, and software or other program to evaluate the ROI image, identify objects, maintain a database of objects, and determine if any objects are potential hazards. FIG. 4 is a block diagram illustrating a system including a computing device 400, a server 425, and an image capture device 485 (e.g., a camera, e.g., the camera system 214 or cameras 314, 315). In various embodiments, fewer, additional and/or different components may be used in the system.

The computing device 400 includes a processor 415 that is coupled to a memory 405. The processor 415 can store and recall data and applications in the memory 405, including applications that process information and send commands/signals according to any of the methods disclosed herein. The processor 415 may also display objects, applications, data, etc. on an interface/display 410 and/or provide an audible alert via a speaker 412. The processor 415 may also or alternately receive inputs through the interface/display 410. The processor 415 is also coupled to a transceiver 420. With this configuration, the processor 415, and subsequently the computing device 400, can communicate with other devices, such as the server 425 through a connection 470 and the image capture device 485 through a connection 480. For example, the computing device 400 may send to the server 425 information determined about a subject from images captured by the image capture device 485, such as depth information of a subject or object in an image.

The server 425 also includes a processor 435 that is coupled to a memory 430 and to a transceiver 440. The processor 435 can store and recall data and applications in the memory 430. With this configuration, the processor 435, and subsequently the server 425, can communicate with other devices, such as the computing device 400 through the connection 470.

The computing device 400 may be, e.g., the computing device 220 of FIG. 2 or the computing device 320 of FIG. 3. Accordingly, the computing device 400 may be located remotely from the image capture device 485, or it may be local and close to the image capture device 485 (e.g., in the same room). The processor 415 of the computing device 400 may perform any or all of the various steps disclosed herein. In other embodiments, the steps may be performed on a processor 435 of the server 425. In some embodiments, the various steps and methods disclosed herein may be performed by both of the processors 415 and 435. In some embodiments, certain steps may be performed by the processor 415 while others are performed by the processor 435. In some embodiments, information determined by the processor 415 may be sent to the server 425 for storage and/or further processing.

The devices shown in the illustrative embodiment may be utilized in various ways. For example, either or both of the connections 470, 480 may be varied. For example, either or both the connections 470, 480 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 470, 480 may be a dock where one device may plug into another device. As another example, one or both of the connections 470, 480 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 470, 480 may represent several different computing devices and network components that allow the various devices to communicate through the internet, either through a hard-wired or wireless connection. One or both of the connections 470, 480 may also be a combination of several modes of connection.

The configuration of the devices in FIG. 4 is merely one physical system on which the disclosed embodiments may be executed. Other configurations of the devices shown may exist to practice the disclosed embodiments. Further, configurations of additional or fewer devices than the ones shown in FIG. 4 may exist to practice the disclosed embodiments. Additionally, the devices shown in FIG. 4 may be combined to allow for fewer devices than shown or separated such that more than the three devices exist in a system. It will be appreciated that many various combinations of computing devices may execute the methods and systems disclosed herein. Examples of such computing devices may include other types of infrared cameras/detectors, night vision cameras/detectors, other types of cameras, radio frequency transmitters/receivers, smart phones, personal computers, servers, laptop computers, tablets, RFID enabled devices, or any combinations of such devices.

The non-contact monitoring systems and methods of this disclosure utilize depth (distance) information between the camera(s) and an object to determine the presence of the object and then determine whether the object poses a threat to a subject. The systems are programmed or trained to recognize different types of objects and their possible positions to determine whether the position of the object poses a threat.

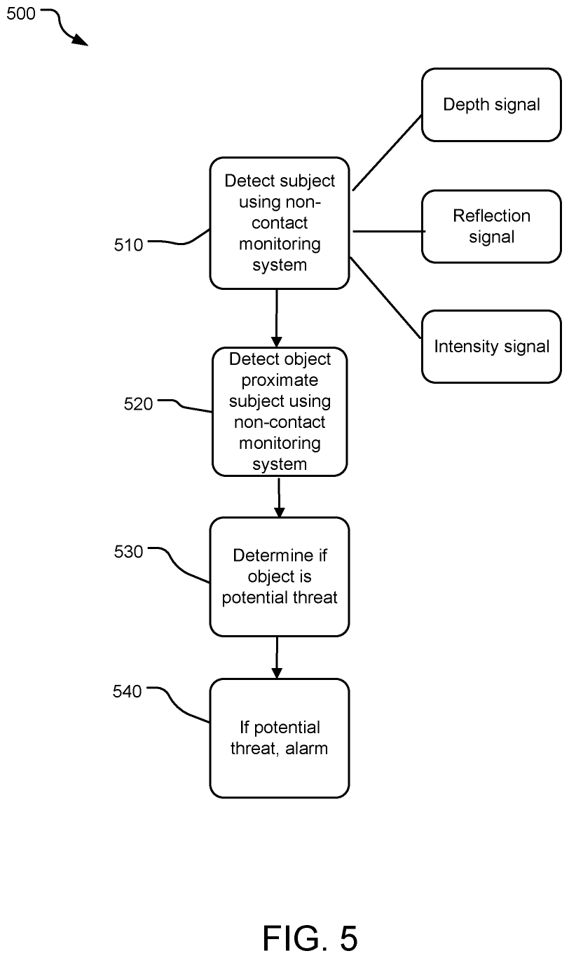

FIG. 5 provides a method 500 for monitoring a subject (e.g., infant, child) using a non-contact monitoring system as described herein. In a first step 510, the location of the subject is detected with the non-contact monitoring system utilizing depth; the depth may be determined from depth measurements, from reflected signals, or from reflected intensity signals, for light, IR, RGB, etc. In a second step 520, the non-contact monitoring system detects an object proximate the subject, in a zone around the subject, and in step 530, the non-contact monitoring system determines if the detected object is a potential hazard to the subject (e.g., by determining if the detected object satisfies one or more hazard criteria). Upon determining the detected object is a potential hazard to the subject, in step 540, the non-contact monitoring system initiates an alarm.

As indicated above, in addition to the methodology of this disclosure utilizing depth (distance) information between the camera(s) and the subject to determine presence and location of an object, the method can also use reflected light intensity from projected light features and/or IR features (e.g., dots, grid, stripes, crosses, squares, etc., or a featureless pattern, or a combination thereof) in the scene to estimate the depth (distance). From the depth information, the presence of an object can be identified in relation to the subject and the system can determine, via AI learning, whether or not the object warrants an alarm to a caregiver.

The above specification and examples provide a complete description of the structure and use of exemplary embodiments of the invention. The above description provides specific embodiments. It is to be understood that other embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

1. A method of monitoring a subject, the method comprising:
detecting a subject with a non-contact monitoring system utilizing depth measurements;
detecting an object within a zone proximate the subject with the non-contact monitoring system utilizing depth measurements;
determining, with the non-contact monitoring system, if the detected object satisfies one or more criteria designating the detected object as a potential hazard to the subject; and
upon determining the detected object satisfies one or more criteria designating the detected object as a potential hazard to the subject, initiating an alarm.

2. The method of claim 1, wherein determining if the detected object satisfies one or more criteria designating the detected object as a potential hazard to the subject comprises:
utilizing artificial intelligence (AI) to determine if the detected object satisfies one or more criteria designating the detected object as a potential hazard to the subject.

3. The method of claim 2, wherein utilizing AI to determine if the detected object satisfies one or more criteria designating the detected object as a potential hazard comprises:
utilizing AI to determine if the detected object is proximate the subject's face.

4. The method of claim 2, wherein utilizing AI to determine if the detected object is proximate the subject's face comprises:
utilizing AI to determine if the detected object is covering some or all of the subject's face.

5. The method of claim 1, wherein determining if the detected object satisfies one or more criteria designating the detected object as a potential hazard to the subject comprises:
utilizing AI to determine the detected object does not satisfy one or more criteria designating the detected object as a potential hazard and is therefore not a potential hazard to the subject.

6. The method of claim 5, wherein utilizing AI to determine if the detected object does not satisfy one or more criteria designating the detected object as a potential hazard comprises:
utilizing AI to determine if the detected object is in a location that is not proximate to the subject's face.

7. The method of claim 1, wherein initiating an alarm comprises:
providing a visual alarm on a display.

8. The method of claim 7, wherein providing a visual alarm on a display comprises:
providing the visual alarm on a device remote from the patient.

9. The method of claim 1, wherein initiating an alarm comprises:
providing an audible alarm.

10. The method of claim 9, wherein providing an audible alarm comprises:
providing the audible alarm from a device remote from the patient.

11. A method of monitoring a subject with a non-contact monitoring system, the method comprising:
detecting a subject in a region of interest (ROI);
detecting an object proximate the subject;
determining, with the non-contact monitoring system, if the detected object satisfies one or more criteria designating the detected object as a potential hazard to the subject, wherein the non-contact monitoring system utilizes artificial intelligence (AI) to determine if the detected object satisfies one or more criteria designating the detected object as a potential hazard; and
upon determining the detected object satisfies one or more criteria designating the detected object as a potential hazard to the subject, initiating an alarm.

12. The method of claim 13, wherein one of the one or more criteria designating the detected object as a potential hazard to the subject is whether the detected object is proximate one or more of the head, face, neck and shoulders of the subject.

13. The method of claim 11, wherein initiating an alarm comprises:
initiating a visual alarm.

14. The method of claim 11, wherein initiating an alarm comprises:
initiating an audible alarm.

15. The method of claim 11, wherein initiating an alarm comprises:
initiating the alarm on a device remote form the non-contact monitoring system.
